(19) United States
(12) Patent Application Publication
(10) Pub. No.: US 2026/0286438 A1
(43) Pub. Date: Sep. 24, 2026

(54) Biomarker composition for predicting efficacy of tuberculosis vaccine comprising NKg7 and use thereof

(71) Applicant: INDUSTRY-ACADEMIC COOPERATION FOUNDATION, YONSEI UNIVERSITY, Seoul (KR)

(72) Inventors: Sang Jun HA, Seoul (KR); Min Chul PARK, Seoul (KR); Dong Jin PARK, Seoul (KR); Sung Jae SHIN, Seoul (KR); Hong Min KIM, Seoul (KR); In Suk LEE, Seoul (KR); Seung Byn BAEK, Seoul (KR); Eun Ho PARK, Seoul (KR); Min Woo PARK, Seoul (KR)

(21) Appl. No.: 19/435,972

(22) Filed: Dec. 30, 2025

(30) Foreign Application Priority Data
Mar. 20, 2025 (KR) 10-2025-0035696

(51) Int. Cl. C12Q 1/6883; C12Q 1/6809; C12Q 1/6851

(52) U.S. Cl. CPC C12Q 1/6883; C12Q 1/6809; C12Q 1/6851; C12Q 2600/106

(57) ABSTRACT

The present invention relates to a method for predicting efficacy of a tuberculosis vaccine comprising NKg7, in which the NKg7 mediates an immune response induced by administration of a tuberculosis vaccine and exhibits increased expression levels in activated immune cells. Accordingly, by measuring expression levels of a NKg7 protein or a gene encoding the same, prediction accuracy for efficacy or protective immunity of a tuberculosis vaccine can be increased, thereby enabling rapid evaluation of vaccine efficacy, and thus the method can be usefully used in the medical field related to tuberculosis vaccines.

### CROSS-REFERENCE TO RELATED APPLICATION

This application is based on and claims priority from Korean Patent Application No. 10-2025-0035696 filed on Marck 20, 2025 in the Korean Intellectual Property Office, the content of which is incorporated herein by reference in its entirety.

### REFERENCE TO AN ELECTRONIC SEQUENCE LISTING

The contents of the electronic sequence listing (JPD20250123US_SEQ.xml; Size: 4,409 bytes; and Date of Creation: Dec. 30, 2025) is herein incorporated by reference in its entirety. The contents of the electronic sequence listing in no way introduces new matter into the specification.

### BACKGROUND

### Field of the Invention

The present invention relates to a method for predicting the efficacy of a tuberculosis vaccine comprising NKg7.

### Description of the Related Art

Tuberculosis is a chronic infectious disease caused by infection with Mycobacterium tuberculosis (MTB), and is the infectious disease that has taken the greatest number of human lives throughout human history. In particular, tuberculosis incidence indicators in Korea (new case incidence rate and mortality rate due to tuberculosis) are the highest among OECD countries. Currently, vaccination with BCG (Bacille Calmette-Guérin) is performed for prevention of tuberculosis; however, the preventive effect thereof ranges from 0 to 80%, shows severe inter-individual variation, and has been reported to have little to no effect in adults or after exposure to Mycobacterium tuberculosis. Therefore, in order to reduce the incidence of tuberculosis to the level of developed countries, development of a new tuberculosis vaccine is essential.

However, repeated failures in tuberculosis vaccine development are attributable to the fact that fundamental research on the pathogenesis of tuberculosis, discovery of new antigens, and the roles of antigens is still very insufficient, such that problems such as development of effective preventive vaccines have not been improved. In particular, although many phenomenological studies have been conducted on how immune cells of a host respond upon infection with Mycobacterium tuberculosis depending on pathogenicity, establishment of animal models for elucidating the mechanisms and knowledge of regulatory mechanisms that have been definitively demonstrated to be beneficial to the host are still very insufficient.

Accordingly, the present inventors completed the present invention by developing a biomarker that can be utilized to predict the efficacy or protective immunity of a tuberculosis vaccine based on mechanisms by which immune cells of a host respond upon infection with Mycobacterium tuberculosis, and a method for predicting protective immunity of a tuberculosis vaccine using the same.

### SUMMARY OF THE INVENTION

An object of the present invention is to provide a biomarker composition for predicting the efficacy of a tuberculosis vaccine comprising natural killer cell granule protein 7 (NKg7).

Another object of the present invention is to provide a composition for predicting the efficacy of a tuberculosis vaccine, comprising a reagent capable of measuring an expression level of a NKg7 protein or a gene encoding the same.

Still another object of the present invention is to provide an information-providing method for predicting the efficacy of a tuberculosis vaccine, comprising: (a) measuring an expression level of a NKg7 protein or a gene encoding the same isolated from a biological sample; and (b) comparing the expression level of the NKg7 protein or the gene encoding the same with a reference level obtained from a control sample.

Still another object of the present invention is to provide a method for screening a tuberculosis vaccine, comprising: (a) administering a tuberculosis vaccine candidate material to a tuberculosis-resistant animal model; (b) measuring an expression level of a NKg7 protein or a gene encoding the same isolated from a biological sample of the animal model to which the candidate material has been administered; and (c) selecting a candidate material in which the expression level of the NKg7 protein or the gene encoding the same is increased compared to a reference level.

In order to achieve the above objects, the present invention provides a biomarker composition for predicting the efficacy of a tuberculosis vaccine comprising NKg7.

In addition, the present invention provides a composition for predicting the efficacy of a tuberculosis vaccine, comprising a reagent capable of measuring an expression level of a NKg7 protein or a gene encoding the same.

In addition, the present invention provides an information-providing method for predicting the efficacy of a tuberculosis vaccine, comprising: (a) measuring an expression level of a NKg7 protein or a gene encoding the same isolated from a biological sample; and (b) comparing the expression level of the NKg7 protein or the gene encoding the same with a reference level obtained from a control sample.

In addition, the present invention provides a method for screening a tuberculosis vaccine, comprising: (a) administering a tuberculosis vaccine candidate material to a tuberculosis-resistant animal model; (b) measuring an expression level of a NKg7 protein or a gene encoding the same isolated from a biological sample of the animal model to which the candidate material has been administered; and (c) selecting a candidate material in which the expression level of the NKg7 protein or the gene encoding the same is increased compared to a reference level.

The composition according to the present invention can increase accuracy of predicting efficacy or protective immunity of a tuberculosis vaccine and enables rapid evaluation of vaccine efficacy, and thus can be usefully used in a medical field related to tuberculosis vaccines.

### BRIEF DESCRIPTION OF THE DRAWINGS

FIG. 1 shows histopathological analysis of lung tissue of female C57BL6 mice at 10 weeks after infection with an M2 strain, which is a tuberculosis strain, and measurement results of CFU (colony forming units) of the tuberculosis strain.

FIG. 2a shows a process for analyzing the degree of activation of CD4⁺ T cells and CD8⁺ T cells, and FIGS. 2b and 2c show degrees of activation of CD4⁺ T cells and CD8⁺ T cells in lung tissue of mice at 1 week, 2 weeks, and 4 weeks after administration of a tuberculosis vaccine to female C57BL6 mice, by flow cytometry analysis.

FIGS. 3a to 3d show results of identifying subtypes of immune cells that increase in response to vaccine administration through single-cell transcriptome analysis by isolating immune cells from lung tissue of mice at 4 weeks, 14 weeks, and 15 weeks after administration of a tuberculosis vaccine to female C57BL6 mice.

FIG. 4 shows results of comparing gene expression patterns of immune cells in lung tissue between a BCG group and a BCG::ESX-1^{Mmar} group through single-cell transcriptome analysis.

FIG. 5a shows results of measuring colony forming units (CFU) of a Mycobacterium tuberculosis strain in the lungs of a tuberculosis-resistant mouse model (C57BL/6) and tuberculosis-susceptible mouse models (A/J and C3H), and FIG. 5b shows results of confirming an association between tuberculosis vaccine efficacy prediction factors and susceptibility to Mycobacterium tuberculosis by comparing, through qRT-PCR, expression changes of genes, including NKg7, whose expression was increased upon administration of BCG::ESX-1Mmar, when the tuberculosis-resistant mouse model (C57BL/6) and the tuberculosis-susceptible mouse models (A/J and C3H) were infected with the M2 strain, which is a tuberculosis strain.

FIG. 6 shows results of comparing expression levels of NKg7 through qRT-PCR in immune cells present in lung and blood at 4 weeks after administration of BCG or BCG::ESX-1^{Mmar} vaccine to female C57BL6 mice.

FIG. 7 shows results of comparing expression levels of NKg7 in immune cells present in lung and blood through a developed qRT-PCR array kit.

### DETAILED DESCRIPTION OF THE INVENTION

Hereinafter, the present invention will be described in detail.

The terms used in the present invention have been selected from general terms that are currently widely used as much as possible while considering the functions of the present invention; however, these may vary depending on the intention of a person skilled in the art to which the present invention pertains or the emergence of new technologies. In addition, in certain cases, arbitrarily selected terms are used, and in such cases, the meanings thereof will be described in detail in the description of the corresponding embodiments. Therefore, the terms used in the present invention should be defined based not on the simple names of the terms, but on the meanings thereof and the contents throughout the present invention.

When a component or a step is described as “comprising” in the present invention, it means that, unless specifically stated otherwise, it does not exclude other components or other steps, but may further include other components or other steps.

### <Composition>

The present invention provides a biomarker composition for predicting the efficacy of a tuberculosis vaccine comprising natural killer cell granule protein 7 (NKg7).

As used herein, the term “tuberculosis vaccine” means a vaccine used to prevent infection caused by Mycobacterium tuberculosis or to prevent severe progression after infection.

The tuberculosis vaccine may be a live vaccine, that is, a strain of the genus Mycobacterium, and preferably may be a recombinant strain of the genus Mycobacterium including a domain capable of inducing an immune response. The domain capable of inducing an immune response is an immunogenic peptide or polypeptide derived from a pathogen or an immunogenic fragment thereof. In another additional embodiment, the vaccine may be a vaccine based on an inactivated or killed whole pathogen strain.

In the present invention, the NKg7 may mediate an immune response induced by a tuberculosis vaccine.

In the present invention, the NKg7 may exhibit increased expression levels in activated immune cells, and the immune cells may be selected from the group consisting of natural killer (NK) cells, CD8⁺ T cells, CD4⁺ T cells, CD8⁺ T cell subtypes, and CD4⁺ T cell subtypes, but are not limited thereto.

In addition, the present invention provides a composition for predicting the efficacy of a tuberculosis vaccine, comprising a reagent capable of measuring an expression level of a NKg7 protein or a gene encoding the same.

In the present invention, the reagent capable of measuring an expression level of the protein may be selected from the group consisting of an antibody, an oligopeptide, a ligand, an aptamer, and a peptide nucleic acid (PNA), each of which specifically binds to the protein or a fragment thereof, but is not limited thereto.

As used herein, the term “antibody” refers to a specific immunoglobulin directed against an antigenic site, and includes polyclonal antibodies, monoclonal antibodies, recombinant antibodies, and combinations thereof. In addition, the term includes not only complete forms having two full-length light chains and two full-length heavy chains, but also functional fragments of antibody molecules, for example, Fab, F(ab′), F(ab′)₂, and Fv. Antibodies can be easily produced using techniques widely known in the art to which the present invention pertains, and commercially available antibodies may be used.

As used herein, the term “aptamer” refers to a type of polynucleotide composed of a special kind of single-stranded nucleic acid (DNA, RNA, or modified nucleic acid) that has a stable three-dimensional structure by itself and is capable of binding to a target molecule with high affinity and specificity. Aptamers can specifically bind to antigenic substances like antibodies, while having higher stability than proteins, simpler structures, and being composed of polynucleotides that are easy to synthesize, and thus can be used as substitutes for antibodies. Aptamers can be easily produced using techniques widely known in the art to which the present invention pertains, and commercially available aptamers may be used.

As used herein, the term “PNA” refers to an artificially synthesized polymer similar to DNA or RNA, wherein DNA has a phosphate-ribose sugar backbone, whereas PNA has a repeating N-(2-aminoethyl)-glycine backbone connected by peptide bonds, thereby greatly increasing binding affinity and stability to DNA or RNA, and thus being used in molecular biology, diagnostic analysis, and antisense therapy. The PNA may be further specified with reference to the literature [Nielsen P E, Egholm M, Berg R H, Buchardt O (December 1991). “Sequence-selective recognition of DNA by strand displacement with a thymine-substituted polyamide”. Science 254(5037): 1497-1500].

In the present invention, the reagent capable of measuring an expression level of the gene may be selected from the group consisting of a primer, a probe, and an antisense oligonucleotide, each of which specifically binds to mRNA of the gene, but is not limited thereto.

Preferably, the reagent capable of measuring an expression level of the gene may be a primer that specifically binds to mRNA of the NKg7 gene, and more preferably may be a primer set comprising the nucleic acid sequences of SEQ ID NOS: 1 and 2, but is not limited thereto.

As used herein, the term “primer” refers to a short nucleic acid sequence having a free 3′-hydroxyl group, which is capable of forming base pairs with a complementary template and acts as a starting point for template strand copying. Under appropriate buffer conditions and temperatures, primers can initiate DNA synthesis in the presence of reagents for polymerization reactions (i.e., DNA polymerase or reverse transcriptase) and four different nucleoside triphosphates. PCR conditions and lengths of sense and antisense primers may be appropriately selected according to techniques known in the art to which the present invention pertains.

As used herein, the term “probe” refers to a nucleic acid fragment such as RNA or DNA capable of specifically binding to a gene, which is labeled so as to identify the presence or absence and expression level of a specific gene. Probes may be prepared in forms such as oligonucleotide probes, single-stranded DNA probes, double-stranded DNA probes, and RNA probes. Selection of appropriate probes and hybridization conditions may be appropriately selected according to techniques known in the art to which the present invention pertains.

As used herein, the term “antisense oligonucleotide” refers to DNA, RNA, or derivatives thereof containing a nucleic acid sequence complementary to a sequence of a specific mRNA, which binds to a complementary sequence within the mRNA and inhibits translation of the mRNA into a protein. An antisense oligonucleotide sequence refers to a DNA or RNA sequence complementary to mRNA of the gene and capable of binding to the mRNA.

### <Kit>

In addition, the present invention provides a kit for predicting the efficacy of a tuberculosis vaccine comprising the composition for predicting the efficacy of a tuberculosis vaccine.

In addition, the present invention provides a kit for use in the method for predicting the efficacy of a tuberculosis vaccine in a subject, comprising a reagent for measuring an expression level of NKg7 or a nucleic acid encoding NKg7.

In the present invention, the kit may comprise a primer set comprising the nucleic acid sequences of SEQ ID NOs: 1 and 2.

In the present invention, the kit may further comprise a primer set comprising the nucleic acid sequences of SEQ ID NOs: 3 and 4, which specifically bind to a reference gene, but is not limited thereto.

In the present invention, the kit may be selected from the group consisting of an RT-PCR kit, a real-time qRT-PCR kit, a DNA chip kit, an ELISA kit, a protein chip kit, and a rapid kit, but is not limited thereto.

In the present invention, the kit may further comprise Oligo (dT), a deoxynucleotide (dNTPs) mix, an RNase inhibitor, DTT (1,4-dithiothreitol), reverse transcriptase, and an RT buffer, but is not limited thereto.

In the present invention, the kit may further comprise a SYBR Green Master Mix and a 96-well PCR array plate, but is not limited thereto.

The RT-PCR kit may include, in addition to respective primer sets specific to marker genes, test tubes or other suitable containers, reaction buffers (with varying pH and magnesium concentrations), deoxynucleotides (dNTPs), enzymes such as Taq polymerase and reverse transcriptase, DNase, RNase inhibitors, DEPC-water, sterile water, and the like. In addition, the kit may include a primer set specific to a gene used as a quantitative control.

The real-time qRT-PCR kit may include respective primer sets and probes specific to marker genes, and may further include test tubes or other suitable containers, reaction buffers (with varying pH and magnesium concentrations), deoxynucleotides (dNTPs), DNA polymerase, DNase, RNase inhibitors, DEPC-water, sterile water, and the like. In addition, the kit may include a primer set specific to a gene used as a quantitative control.

The DNA chip kit may include a substrate on which cDNA corresponding to a gene or a fragment thereof is attached as a probe, and reagents, formulations, enzymes, and the like for producing fluorescently labeled probes. In addition, the substrate may include cDNA corresponding to a quantitative control gene or a fragment thereof.

The ELISA kit may include monoclonal antibodies, polyclonal antibodies, or recombinant antibodies specific to a protein, and may further include reagents capable of detecting bound antibodies, for example, labeled secondary antibodies, chromophores, enzymes (e.g., conjugated to antibodies) and substrates thereof, or other substances capable of binding to antibodies.

The protein chip kit may include, for immunological detection of antibodies, substrates, appropriate buffers, secondary antibodies labeled with chromogenic enzymes or fluorescent substances, chromogenic substrates, and the like. The substrates may include nitrocellulose membranes, 96-well plates made of polyvinyl resin, 96-well plates made of polystyrene resin, and glass slide glasses, and the chromogenic enzymes may include peroxidase and alkaline phosphatase, the fluorescent substances may include FITC and RITC, and the chromogenic substrate solutions may include ABTS (2,2′-azino-bis(3-ethylbenzothiazoline-6-sulfonic acid)), OPD (o-phenylenediamine), and TMB (tetramethylbenzidine).

The rapid kit is a kit capable of rapidly performing a test using a small amount of a specimen, and may use a substance capable of binding to an analyte contained in a sample, that is, an antibody capable of specifically binding to a protein.

### Method

The present invention provides a method for predicting the efficacy of a tuberculosis vaccine in a subject, comprising: (a) measuring an expression level of natural killer cell granule protein 7 (NKg7) or a nucleic acid encoding NKg7 in a biological sample obtained from the subject; and (b) comparing the measured expression level with a reference level obtained from a control sample.

Descriptions overlapping with those described above are omitted.

In the present invention, the expression level of NKg7 or the nucleic acid encoding NKg7 may be associated with an immune response induced by administration of a tuberculosis vaccine.

In the present invention, the expression level of NKg7 or the nucleic acid encoding NKg7 may be increased in activated immune cells relative to non-activated immune cells.

In the present invention, the biological sample may be selected from the group consisting of tissue, cells, blood, serum, plasma, lymph, saliva, and urine, and preferably may be lung tissue, blood, serum, or plasma, but is not limited thereto.

In the present invention, the method may further comprises determining that the tuberculosis vaccine has a high efficacy when the expression level of the NKg7 or the nucleic acid encoding NKg7 is higher than the reference level obtained from the control sample.

In the present invention, the mRNA expression level may be measured using reverse transcription polymerase chain reaction (RT-PCR), competitive reverse transcription polymerase chain reaction (competitive RT-PCR), real-time quantitative reverse transcription polymerase chain reaction (real-time quantitative RT-PCR), RNase protection method, Northern blotting, or DNA chip technology, and the protein expression level may be measured using western blotting, enzyme-linked immunosorbent assay (ELISA), radioimmunoassay (RIA), radial immunodiffusion, Ouchterlony immunodiffusion, rocket immunoelectrophoresis, immunohistochemical staining, immunoprecipitation assay, complement fixation assay, immunofluorescence, immunochromatography, fluorescence-activated cell sorter (FACS) analysis, or protein chip technology.

The present invention provides a method for screening a tuberculosis vaccine candidate, comprising: (a) administering a tuberculosis vaccine candidate to a tuberculosis-resistant animal model; (b) measuring an expression level of NKg7 or a nucleic acid encoding NKg7 in a biological sample obtained from the animal model; and (c) selecting the tuberculosis vaccine candidate when the expression level is increased relative to a reference level.

In the present invention, the tuberculosis-resistant animal model may be an animal model exhibiting enhanced immune resistance to Mycobacterium tuberculosis infection compared to a non-immunized control.

In the present invention, the tuberculosis-resistant animal model may be selected from the group consisting of mice, guinea pigs, rats, rabbits, and non-human primates, but is not limited thereto.

In the present invention, the candidate material may be administered as a single dose or multiple doses, and in the case of multiple doses administrations, may be administered 2 to 5 times, but is not limited thereto.

In the present invention, the step of measuring an expression level of the NKg7 protein or the gene encoding the same may be performed at 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks after administration of the vaccine, and preferably may be performed at 1 week, 4 weeks, 8 weeks, 12 weeks, 14 weeks, or 15 weeks after administration, but is not limited thereto.

In the present invention, the biological sample may be selected from the group consisting of tissue, cells, blood, serum, plasma, lymph, saliva, and urine, but is not limited thereto.

In the present invention, the method may further comprise measuring expression levels of the NKg7 protein or the gene encoding the same before and after administration of the candidate material.

### EXAMPLES

The present invention will be described in further detail through the following examples. However, these examples are provided for illustrative purposes only and are not intended to limit the scope of the invention in any way.

### Example 1. Evaluation of Protective Efficacy of Tuberculosis Vaccine in Mouse Animal Model

An experiment was performed to compare protective efficacy of an existing BCG vaccine and a recombinant BCG vaccine (BCG::ESX-1^{Mmar}) in a mouse animal model. Briefly, female C57BL6 mice were administered with the existing BCG vaccine or the recombinant BCG vaccine (BCG::ESX-1^{Mmar}), and after 10 weeks, were infected with an M2 strain, which is a tuberculosis strain. Thereafter, at 10 weeks after infection with the tuberculosis strain, histopathological analysis of lung tissue of the mice and measurement of CFU (colony forming units) of the tuberculosis strain in the lung and spleen were performed. The experimental groups were composed of a Naive group without infection; an Infection group infected with the tuberculosis strain without vaccine administration; a BCG group infected with the tuberculosis strain after prior administration of the BCG vaccine 10 weeks before the infection time point; and a BCG::ESX-1^{Mmar} group infected with the tuberculosis strain after prior administration of the BCG::ESX-1^{Mmar} vaccine 10 weeks before the infection time point.

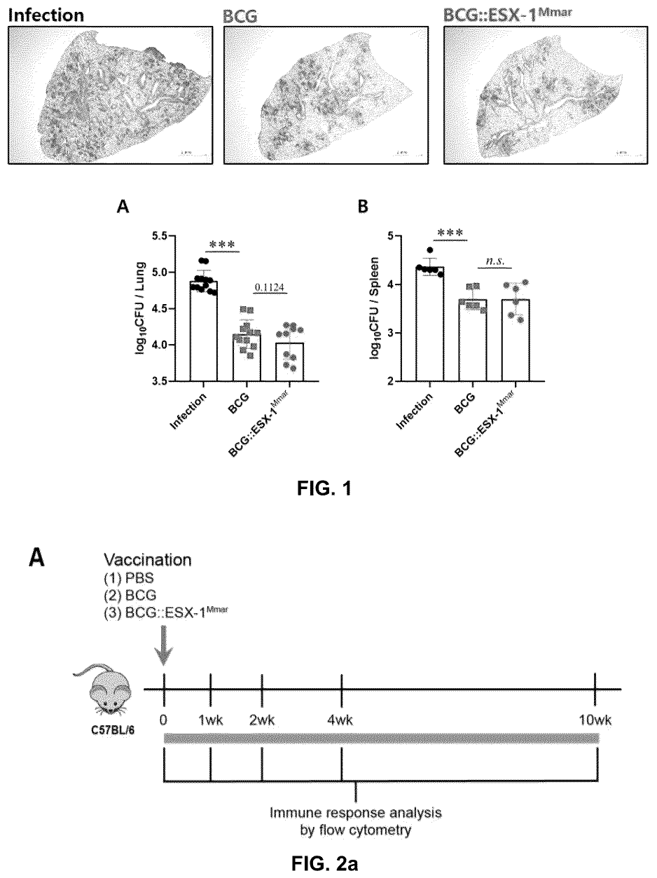

As a result, as shown in FIG. 1, in the BCG group and the BCG::ESX-1^{Mmar} group, severity of lung tissue damage caused by the tuberculosis strain was favorably recovered compared to the Infection group, and CFU of the tuberculosis strain in the lung and spleen was also significantly reduced. In particular, in the BCG::ESX-1^{Mmar} group, lung tissue recovery was observed to be further improved compared to the BCG group, and CFU of the tuberculosis strain in the lung was also confirmed to be further reduced.

From the above results, in the present invention, it was confirmed that the BCG vaccine or the recombinant BCG vaccine has tuberculosis preventive effects such as lung tissue recovery effects and reduction effects of Mycobacterium tuberculosis, and in particular, that the recombinant BCG vaccine, BCG::ESX-1^{Mmar}, has higher protective efficacy or protective ability than the existing BCG vaccine.

### Example 2. Confirmation of T Cell Activation Induction Ability by Tuberculosis Vaccine in Mouse Animal Model

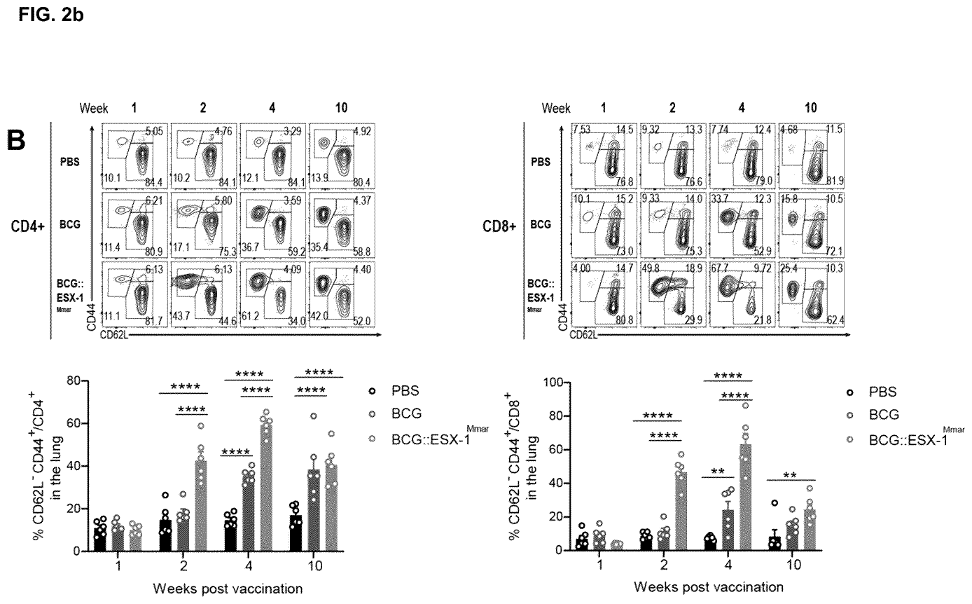

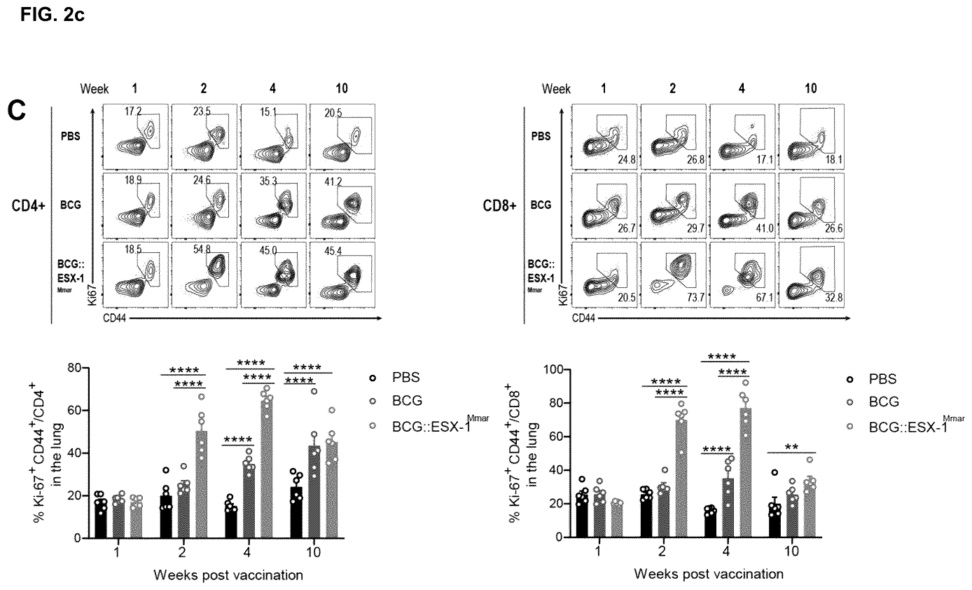

An experiment was performed to compare T cell activation induction ability by an existing BCG vaccine and a recombinant BCG vaccine in a mouse animal model. Briefly, female C57BL6 mice were administered with the existing BCG vaccine or the recombinant BCG vaccine, and immune cells isolated from lung tissue of the mice were analyzed for degrees of activation of CD4⁺ T cells and CD8⁺ T cells by flow cytometry at 1 week, 2 weeks, 4 weeks, and 10 weeks after vaccine administration (FIG. 2A). The experimental groups were composed of a PBS group to which no vaccine was administered; a group administered with the BCG vaccine; and a group administered with the BCG::ESX-1^{Mmar} vaccine. Statistical significance was evaluated by a Two-Way ANOVA test.

As a result, as shown in FIGS. 2B and 2C, in the BCG group and the BCG::ESX-1^{Mmar} group, activation of CD4⁺ T cells and CD8⁺ T cells in lung tissue was confirmed to be markedly induced compared to the PBS group to which no vaccine was administered. In particular, in the BCG group, from 4 weeks after vaccine administration, proportions of effector T cells and proportions of proliferating T cells were significantly increased. On the other hand, in the BCG::ESX-1^{Mmar} group, proportions of T cells were markedly increased from 2 weeks after vaccine administration, and it was phenotypically confirmed that activation of T cells was all significantly higher than that in the BCG group until 10 weeks, which is before the immune response induced by the vaccine ends.

From the above results, in the present invention, it was confirmed that the BCG vaccine or the BCG::ESX-1^{Mmar} vaccine induces T cell activation, and in particular, that the BCG::ESX-1^{Mmar} vaccine is capable of inducing strong activation of T cells from an earlier time point compared to the existing BCG vaccine.

### Example 3. Identification of T Cell Subtypes Increased in Response to Tuberculosis Vaccine Through Single-Cell Transcriptome Analysis

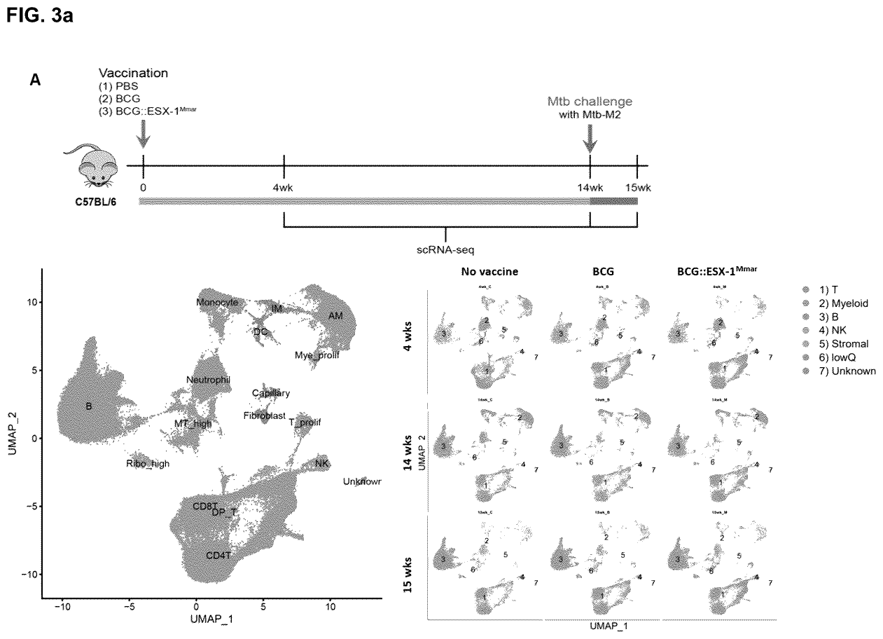

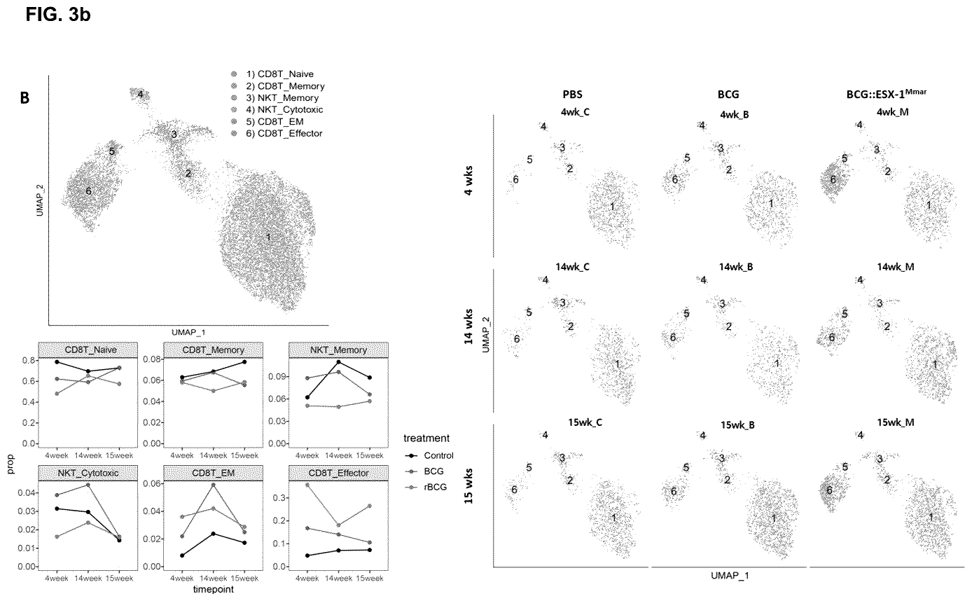

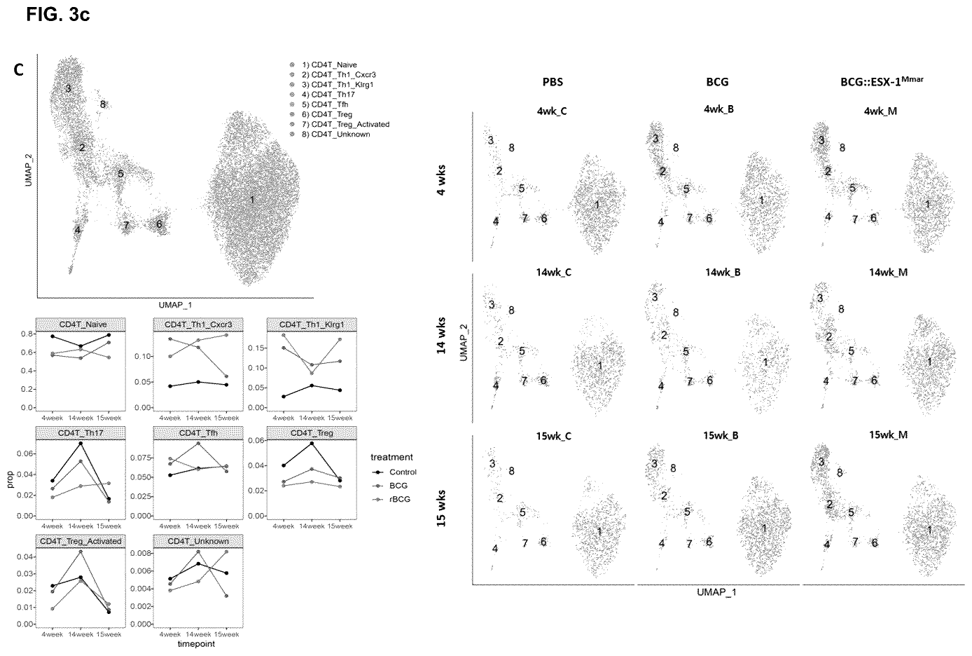

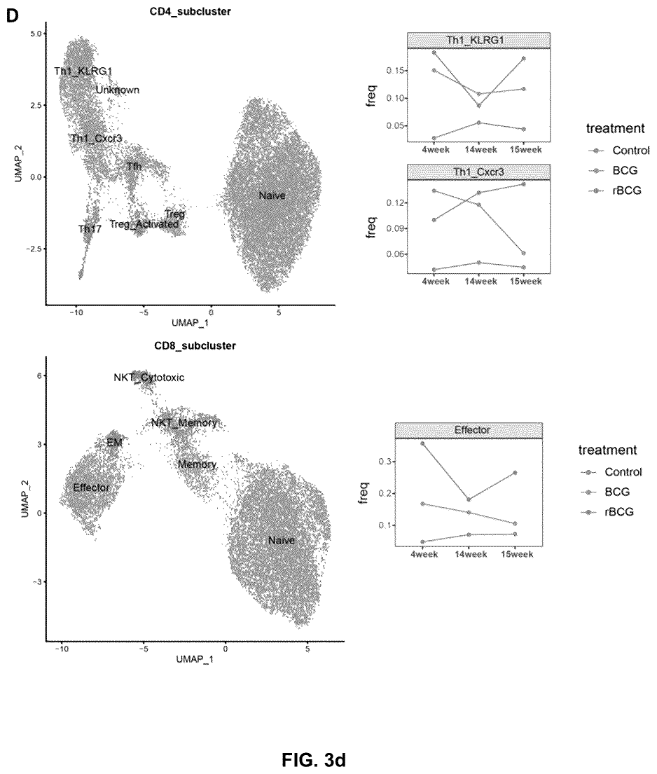

An experiment was performed to identify T cell subtypes that increase in response to a tuberculosis vaccine in a mouse animal model. Briefly, female C57BL6 mice were administered with an existing BCG vaccine or a BCG: ESX-1^{Mmar} vaccine, and immune cells were isolated from lung tissue of the mice at 4 weeks, 14 weeks, and 15 weeks after vaccine administration, respectively, followed by performing single-cell transcriptome analysis (scRNA-seq), and immune responses induced by the tuberculosis vaccine were compared by immune cell subtype units (FIG. 3A). At 14 weeks after vaccine administration, infection was performed with an M2 strain, which is a tuberculosis strain. The experimental groups were composed of a PBS group to which no vaccine was administered; a BCG group administered with the BCG vaccine; and a BCG::ESX-1^{Mmar} group administered with the BCG::ESX-1^{Mmar} vaccine.

As a result, it was confirmed that CD4⁺ T cells and CD8⁺ T cells among immune cells increased upon administration of the tuberculosis vaccine (FIGS. 3B and 3C). In addition, in the group administered with the BCG::ESX-1^{Mmar} vaccine, it was confirmed that, among CD8⁺ T cell subtypes, a proportion of effector CD8⁺ T cell subtypes was markedly increased compared to the BCG group, and that, among CD4⁺ T cell subtypes, proportions of two CD4⁺ Th1 cell subtypes (KLRG1⁺ and CXCR3⁺ Th1), which have been reported to play important roles in bacterial response, were markedly increased. In particular, differences between the BCG group and the BCG::ESX-1^{Mmar} group in T cell subtype proportions were clearly observed in samples at 4 weeks after vaccine administration and at 15 weeks after vaccine administration (1 week after infection with the M2 strain) (FIG. 3D).

From the above results, in the present invention, it was confirmed that proportions of specific subtypes within CD4⁺ T cells and CD8⁺ T cells show an increasing tendency in the BCG::ESX-1^{Mmar} group compared to the BCG group.

### Example 4. Comparison of Gene Expression Patterns Induced by Administration of Recombinant BCG Tuberculosis Vaccine

Based on the single-cell transcriptome analysis (scRNA-seq) data of Example 3, an experiment was performed to compare gene expression patterns of immune cells in lung tissue between a BCG group and a BCG::ESX-1^{Mmar} group in order to identify factors contributing to high efficacy or protective immunity against the BCG::ESX-1^{Mmar} vaccine.

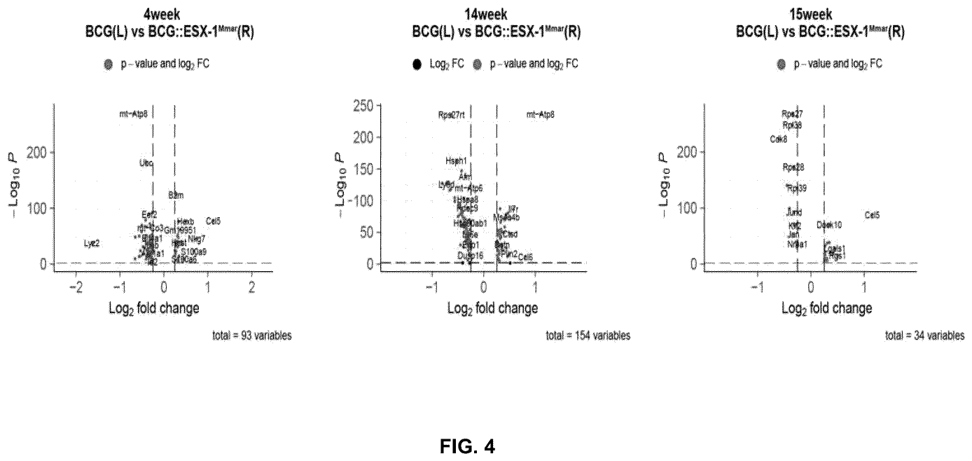

As a result, as shown in FIG. 4, nine genes whose expression levels were relatively increased in the BCG::ESX-1^{Mmar} group were secured as a candidate group of efficacy prediction factors, and these are shown in Table 1 below. In addition, among the nine genes, the gene exhibiting the greatest increase in expression level was identified as natural killer cell granule protein 7 (NKg7).

TABLE 1

| | p_val | | | | | | |
|---|---|---|---|---|---|---|---|
| | (BCG vs | | | | | | |
| Gene | BCG::ESX- | average log2 | | | | time | cell |
| (Accession No.) | 1^{Mmar}) | fold change | pct.1 | pct.2 | p_val_adj | point | type |
| Nkg7 | 4.46E−51 | 0.744766698 | 0.29 | 0.204 | 1.23E−46 | 4 week | Total |
| (NM_024253) | | | | | | | |
| S100a9 | 6.14E−28 | 0.67770128 | 0.29 | 0.22 | 1.70E−23 | 4 week | Total |
| (NM_001281852) | | | | | | | |
| Hexb | 1.69E−81 | 0.493985618 | 0.904 | 0.889 | 4.68E−77 | 4 week | Total |
| (NM_010422) | | | | | | | |
| S100a6 | 5.76E−14 | 0.460707446 | 0.36 | 0.313 | 1.59E−09 | 4 week | Total |
| (NM_011313) | | | | | | | |
| Crip1 | 3.10E−14 | 0.436807072 | 0.504 | 0.47 | 8.57E−10 | 4 week | Total |
| (NM_007763) | | | | | | | |
| Il18rap | 2.43E−40 | 0.397678027 | 0.28 | 0.199 | 6.72E−36 | 4 week | Total |
| (NM_010553) | | | | | | | |
| Hcst | 3.32E−44 | 0.342178796 | 0.612 | 0.548 | 9.18E−40 | 4 week | Total |
| (NM_011827) | | | | | | | |
| Dusp2 | 3.70E−19 | 0.340899794 | 0.48 | 0.428 | 1.02E−14 | 4 week | Total |
| (NM_010090) | | | | | | | |
| Pglyrp1 | 1.13E−12 | 0.29566299 | 0.261 | 0.216 | 3.13E−08 | 4 week | Total |
| (NM_009402) | | | | | | | |

### Example 5. Verification of Tuberculosis Vaccine Efficacy Prediction Factor Candidates Based on Tuberculosis-Resistant or Tuberculosis-Susceptible Mouse Animal Models

An experiment was performed to verify candidate groups of tuberculosis vaccine efficacy prediction factors in mouse animal models having different susceptibilities to Mycobacterium tuberculosis. Briefly, after mice were infected with an M2 strain, which is a tuberculosis strain, RNA was isolated from lung tissue of the mice at 3 weeks and 4 weeks after infection with Mycobacterium tuberculosis, and expression levels of genes of candidate groups of tuberculosis vaccine efficacy prediction factors, including NKg7, were compared through qRT-PCR. The experimental groups were composed of a Mycobacterium tuberculosis-resistant (or low-susceptibility) C57BL6 mouse group; a Mycobacterium tuberculosis-high-susceptibility A/J mouse group; and a Mycobacterium tuberculosis-high-susceptibility C3H mouse group (FIG. 8A). The primer sets used are shown in Table 2 below.

TABLE 2

| Gene | Accession | Forward Primer (5′ → 3′) | Size | Tm | GC | Size |
|---|---|---|---|---|---|---|
| symbol | No. | Reverse Primer (5′ → 3′) | (bp) | (° C.) | (%) | (bp) |
| NKg7 | NM_024253 | CCAACAAGCCAAGAGACTCA | 20 | 54.9 | 50 | 75 |
| | | (SEQ ID NO: 1) | | | | |
| | | CCACAGGACAGCCAGGATAC | 20 | 57.5 | 60 | |
| | | (SEQ ID NO: 2) | | | | |
| β-actin | NM 007393.3 | AGTGCTTCTAGGCGGACTGTTA | 22 | 65 | 50 | 70 |
| | | (SEQ ID NO: 3) | | | | |
| | | TTTCTGCGCAAGTTAGGTTTT | 21 | 61.4 | 38.1 | |
| | | (SEQ ID NO: 4) | | | | |

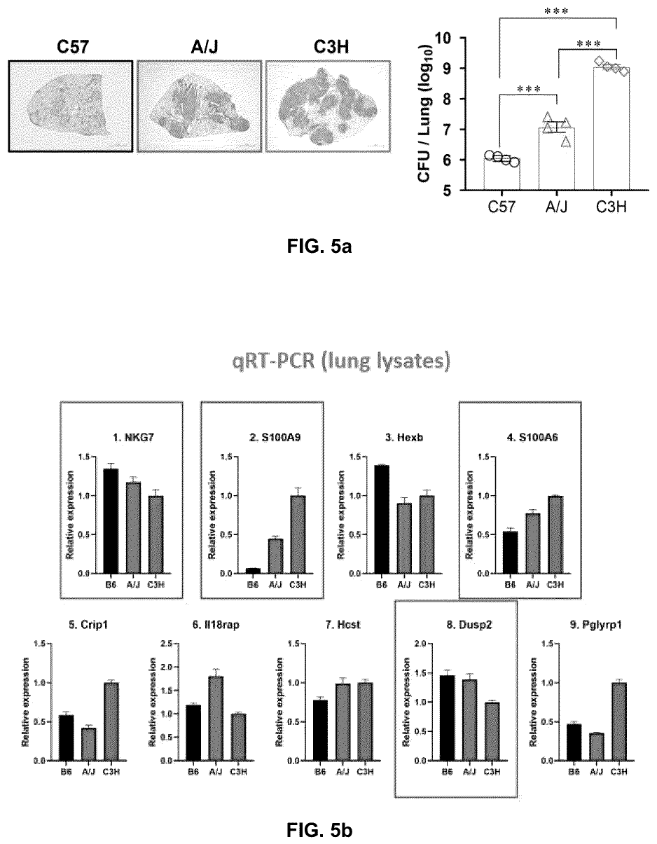

As a result, as shown in FIG. 5B, in the Mycobacterium tuberculosis-resistant (or low-susceptibility) C57BL6 mouse group, expression levels of NKg7 were confirmed to be increased by a large extent after infection with Mycobacterium tuberculosis. In contrast, in the Mycobacterium tuberculosis-high-susceptibility C3H and A/J mouse groups, it was confirmed that the extent of increase in NKg7 expression levels was relatively low.

From the above results, it was confirmed that the lower the susceptibility of mice to Mycobacterium tuberculosis, the greater the increase in NKg7 expression levels, and thus, it was confirmed that NKg7 can be used as an efficacy prediction factor of a tuberculosis vaccine for screening of a tuberculosis vaccine in Mycobacterium tuberculosis-resistant or low-susceptibility animal models.

### Example 6. Comparison of Changes in NKg7 Expression Levels According to Tuberculosis Vaccines

An experiment was performed to compare changes in NKg7 expression levels according to administration of tuberculosis vaccines in immune cells present in lung and blood of a mouse animal model. Briefly, female C57BL6 mice were administered with a BCG vaccine or a BCG: ESX-1^{Mmar} vaccine, and at 4 weeks after vaccine administration, RNA was isolated from immune cells present in lung and blood, and NKg7 expression levels were compared through qRT-PCR. The experimental groups were composed of a PBS group to which no vaccine was administered; a BCG group administered with the BCG vaccine; and a BCG: ESX-1^{Mmar} group administered with the BCG::ESX-1^{Mmar} vaccine. Statistical significance was evaluated by a One-Way ANOVA test.

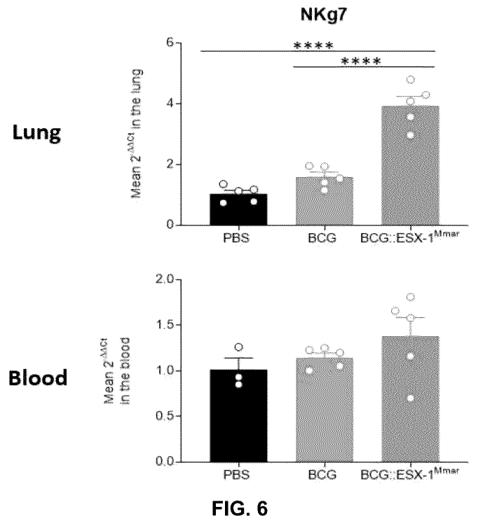

As a result, as shown in FIG. 6, it was confirmed that NKg7 expression levels were markedly increased in the BCG::ESX-1^{Mmar} group compared to the PBS group or the BCG group. This tendency was similarly observed not only in immune cells isolated from lung tissue but also in immune cells isolated from blood.

From the above results, it was confirmed that the BCG::ESX-1^{Mmar} vaccine has higher protective efficacy or protective immunity than the existing BCG vaccine, and by confirming that NKg7 expression levels were higher in a group administered with the BCG::ESX-1^{Mmar} vaccine having higher protective efficacy, it was confirmed that efficacy of a tuberculosis vaccine can be predicted by measuring expression levels of a NKg7 protein or a gene encoding the same in immune cells isolated from lung tissue or blood.

### Example 7. Validation of Effectiveness of qRT-PCR Array Kit

In the present invention, a kit for predicting efficacy of a tuberculosis vaccine including a primer set capable of measuring NKg7 expression levels was developed, and an experiment was performed to validate effectiveness of the developed qRT-PCR kit. The tuberculosis vaccine efficacy prediction kit was prepared to measure NKg7, which is an efficacy evaluation indicator gene, and β-actin was selected as a reference gene. The kit was configured such that expression levels of a target gene, NKg7, and a reference gene, β-actin, are simultaneously measured in one 96-well plate, and three types of samples, a negative control, a positive control, and a test sample, are subjected to triplicate experiments to obtain statistically significant results. In addition, in order to simplify a process of reverse transcription of total RNA extracted from samples prior to performing a PCR array, reagents used at each step were designed in a pre-mixture form. The primer sequences used are the same as those in Table 2 above, and components of the kit are shown in Table 3 below.

TABLE 3
1. Reverse Transcription Component

| | | | |
|---|---|---|---|
| RT I. Reagent | 60 | μL | Oligo (dT) + dNTP Mix |
| RT II. Reagent | 200 | μL | RT Buffer + DTT + RNase Inhibitor |
| RT III. Reagent | 30 | μL | Reverse Transcriptase |

2. PCR Array Component

| | | | |
|---|---|---|---|
| PCR Master Mix | 300 | μL | SYBR Green Master Mix |
| PCR Array Plate | 3 | plates | 96-well PCR Array Plate |

In the same manner, as in Example 6 above, female C57BL6 mice were administered with a BCG vaccine or a BCG::ESX-1^{Mmar} vaccine, and at 4 weeks after vaccine administration, RNA was isolated from immune cells present in lung and blood. Thereafter, NKg7 expression levels were compared using the qRT-PCR array kit. The experimental groups were composed of a PBS group to which no vaccine was administered; a group administered with the BCG vaccine; and a BCG::ESX-1^{Mmar} group administered with the BCG::ESX-1^{Mmar} vaccine. Statistical significance was evaluated by a One-Way ANOVA test.

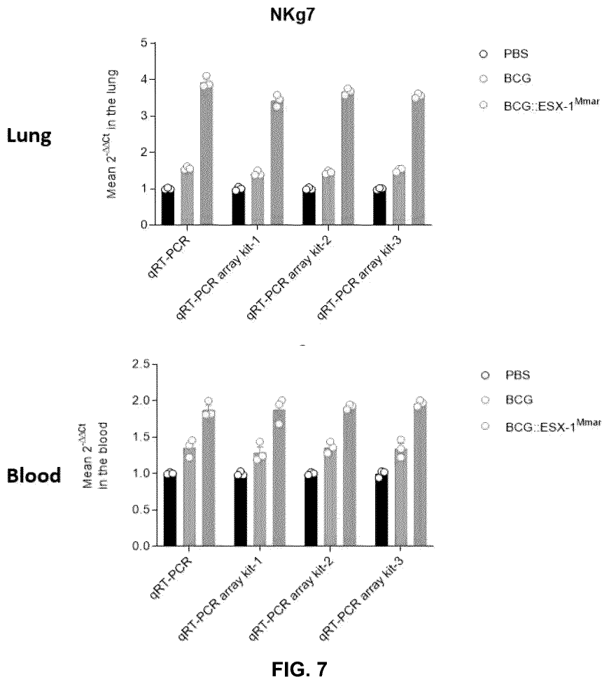

As a result, as shown in FIG. 7, it was confirmed that the developed qRT-PCR array kit exhibited higher NKg7 expression levels in the group administered with the BCG::ESX-1^{Mmar} vaccine, consistently with qRT-PCR results.

Accordingly, in the present invention, it was confirmed that a kit including a reagent capable of measuring expression levels of a NKg7 protein or a gene encoding the same can be usefully used for predicting efficacy of a tuberculosis vaccine.

The above description of the present invention is for illustrative purposes, and a person skilled in the art to which the present invention pertains will understand that various modifications can be easily made into other specific forms without changing the technical spirit or essential characteristics of the present invention. Therefore, the embodiments described above should be understood as illustrative in all respects and not restrictive. For example, each component described in a singular form may be implemented in a distributed form, and likewise, components described in a distributed form may be implemented in a combined form.

The scope of the present invention is defined by the appended claims, and all changes or modifications derived from the meaning and scope of the claims and equivalents thereof should be interpreted as being included in the scope of the present invention.

What is claimed is:

1. A method for predicting the efficacy of a tuberculosis vaccine in a subject, comprising:
(a) measuring an expression level of natural killer cell granule protein 7 (NKg7) or a nucleic acid encoding NKg7 in a biological sample obtained from the subject; and
(b) comparing the measured expression level with a reference level obtained from a control sample.

2. The method of claim 1,
wherein the expression level of NKg7 or the nucleic acid encoding NKg7 is associated with an immune response induced by administration of a tuberculosis vaccine.

3. The method of claim 1,
wherein the expression level of NKg7 or the nucleic acid encoding NKg7 is increased in activated immune cells relative to non-activated immune cells.

4. The method of claim 3,
wherein the immune cells are selected from the group consisting of natural killer (NK) cells, CD8⁺ T cells, CD4⁺ T cells, CD8⁺ T cell subtypes, and CD4⁺ T cell subtypes.

5. The method of claim 1,
wherein a reagent for measuring the expression level of the NKg7 comprises an antibody, an oligopeptide, a ligand, an aptamer, or a peptide nucleic acid (PNA), each specifically binding to NKg7 or a fragment thereof.

6. The method of claim 1,
wherein a reagent for measuring the expression level of the nucleic acid encoding NKg7 is a primer, a probe, or an antisense oligonucleotide, each of which specifically binds to mRNA of the nucleic acid encoding NKg7.

7. The method of claim 1,
wherein the biological sample is selected from the group consisting of tissue, cells, whole blood, serum, plasma, lymph, saliva, and urine.

8. The method of claim 1,
further comprising determining that the tuberculosis vaccine has a high efficacy when the expression level of the NKg7 or the nucleic acid encoding NKg7 is higher than the reference level obtained from the control sample.

9. A kit for use in the method of claim 1, comprising a reagent for measuring an expression level of NKg7 or a nucleic acid encoding NKg7.

10. The kit of claim 9,
wherein the kit is selected from the group consisting of an RT-PCR kit, a real-time qRT-PCR kit, a DNA microarray kit, an ELISA kit, a protein microarray kit, and a rapid diagnostic kit.

11. A method for screening a tuberculosis vaccine candidate, comprising:
(a) administering a tuberculosis vaccine candidate to a tuberculosis-resistant animal model;
(b) measuring an expression level of NKg7 or a nucleic acid encoding NKg7 in a biological sample obtained from the animal model; and
(c) selecting the tuberculosis vaccine candidate when the expression level is increased relative to a reference level.

12. The method of claim 11,
wherein the tuberculosis-resistant animal model is an animal model exhibiting an enhanced immune resistance to Mycobacterium tuberculosis infection compared to a non-immunized control.

13. The method of claim 11,
the tuberculosis-resistant animal model is selected from the group consisting of mice, guinea pigs, rats, rabbits, and non-human primates.

14. The method of claim 11,
wherein the tuberculosis vaccine candidate is administered as a single dose or multiple doses.

15. The method of claim 11,
wherein the biological sample is selected from the group consisting of tissue, cells, whole blood, serum, plasma, lymph, saliva, and urine.

16. The method of claim 11,
further comprising measuring the expression level of NKg7 or the nucleic acid encoding NKg7 before and after administration of the tuberculosis vaccine candidate.


SEQUENCE LISTING (4 sequences)

SEQ ID NO: 1 - DNA, 20 bp, synthetic construct
  source 1..20: mol_type = other DNA
ccaacaagccaagagactca

SEQ ID NO: 2 - DNA, 20 bp, synthetic construct
  source 1..20: mol_type = other DNA
ccacaggacagccaggatac

SEQ ID NO: 3 - DNA, 22 bp, synthetic construct
  source 1..22: mol_type = other DNA
agtgcttctaggcggactgtta

SEQ ID NO: 4 - DNA, 21 bp, synthetic construct
  source 1..21: mol_type = other DNA
tttctgcgcaagttaggtttt